(19) United States
(12) Patent Application Publication
(10) Pub. No.: US 2009/0123995 A1
(43) Pub. Date: May 14, 2009

(54) WASTE TREATMENT SYSTEM

(75) Inventor: Loris Bressan, Imola (IT)

Correspondence Address: VOLPE AND KOENIG, P.C., UNITED PLAZA, SUITE 1600, 30 SOUTH 17TH STREET, PHILADELPHIA, PA, 19103, US

(73) Assignee: AMBIENTALIA S.r.l., Bologna (IT)

(21) Appl. No.: 11/914,212

(22) PCT Filed: Apr. 28, 2006
(86) PCT No.: PCT/IB2006/001048

§ 371 Date: Jun. 26, 2008

(30) Foreign Application Priority Data
May 13, 2005 (IT) BO2005A000343

(51) Int. Cl. C12M 1/00

(52) U.S. Cl. 435/293.1

(57) ABSTRACT

A waste treatment system comprising an impermeable container adapted to accommodate waste, and presenting at least an opening for introducing waste and a plurality of preferential breaking lines for creating respective openings; air-tight closing element of the opening; percolate introduction element within the impermeable container; collector for the formed biogas; a covering layer arranged so as to close the openings; and an air circulation system comprising in turn, within the permeable container a plurality of intake tubes and a plurality of air intakes.

### FIELD OF INVENTION

The present invention relates to a waste treatment system.

### BACKGROUND

The problem of waste disposal or recycling has been particularly felt for some time. In particular, the biggest problems of organic waste are related to its high environmental impact.

With regard to this, new European regulations related to waste disposal indicate sanitation and composting as particularly valid solutions.

Composting is a natural form of organic waste disposal known for a long time, by means of which a product, compost, is made and used in agriculture as soil conditioner, as fertiliser or as cultivation substrate.

In particular, composting is a bioxidising exothermal process which occurs in controlled conditions and, by means of the action of micro organisms, leads to the production of water, carbon dioxide, heat and compost.

Another form of organic waste exploitation relates to the production of biogas by anaerobic fermentation. The produced biogas may be either converted into electricity and/or heat in a cogeneration system or be introduced directly into the gas network.

In order to exploit both the potentials of biogas and those of compost, the organic waste recycling process should envisage two consequential steps: a first step in which the organic waste is subjected to an aerobic treatment for biogas formation, and a second step in which the waste is subjected to aerobic treatment for the preparation of compost.

As apparent, the passage from the first to the second step may entail a series of problems above all of environmental and logistic nature. Indeed, the transportation of waste from an anaerobic reactor to an aerobic reactor is particularly complex and, obviously, environmentally hazardous.

### SUMMARY

It is the object of the present invention to make a system for the treatment of organic waste which allows to simply and cost-effectively implement a complete recycling process performed either in only aerobic or in combined aerobic and anaerobic conditions, without presenting logistic or environmental problems.

The object of the present invention is a waste treatment system comprising a permeable material container adapted to accommodate the waste, and presenting at least one opening for introducing the waste, air-tight closing element of said opening, percolate introduction element within the permeable material container, a collector for the formed biogas, a covering layer made of adsorbing and transpiring material arranged at an upper portion of said container, and a recirculation system of the air within said container.

### BRIEF DESCRIPTION OF THE DRAWINGS

The following example is provided by way of non-limiting illustration for better understanding of the invention with the help of the figures in the accompanying drawing, in which:

FIG. 1 is a side section with schematised parts of a preferred embodiment of the system according to the present invention;

FIG. 2 is a cross-section of the system shown in FIG. 1;

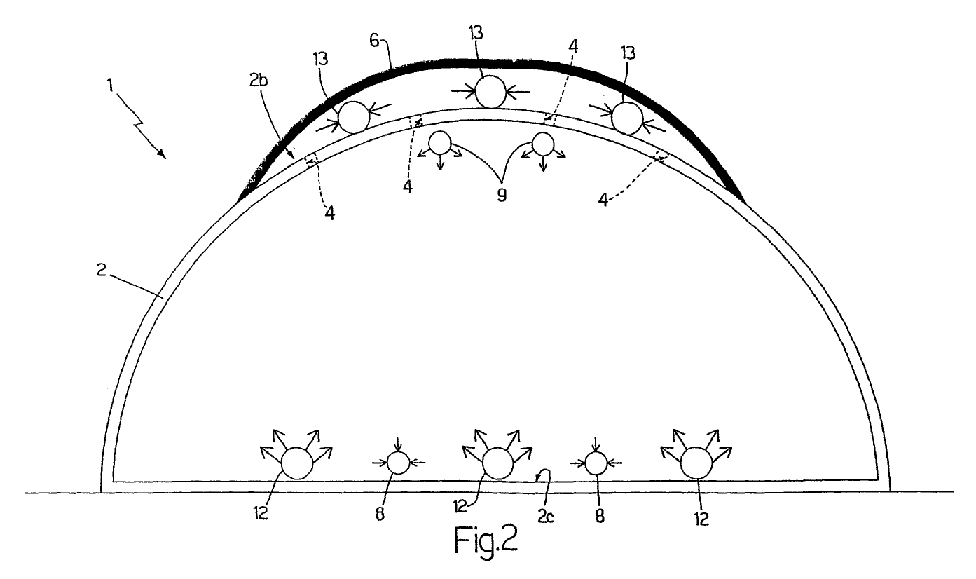

FIG. 3 is a side section with schematised parts of a second preferred embodiment of the system according to the present invention; and

FIG. 4 is a cross-section of the system shown in FIG. 3.

### DETAILED DESCRIPTION OF THE PREFERRED EMBODIMENTS

In the figure, it is shown as a whole by the waste treatment system object of the present invention.

System 1 comprises a tubular container 2 made of polyethylene or other suitable material, presenting on one end 2a an opening 3 for introducing waste. The tubular container 2 is therefore made of non-rigid material for facilitating transportation, installation and removal thereof.

System 1 comprises closing element 5 (known and not described in general) for the opening 3, and an adsorbing and transpiring layer 6 arranged at the upper position 2b for closing the openings 4 formed as described below.

The adsorbing and transpiring layer 6 is formed by an open cell polyurethane matrix impregnated with activate carbon or other filtering system. The adsorbing and transpiring layer 6 has mainly the purpose of blocking odorous substances and filtering them before they are dispersed in the environment and of attenuating the output of bad odours caused by the decomposition of waste during compositing. The function of the adsorbing and transpiring layer 6 is also to adsorb part of the output humidity to gradually give it to the decomposing material.

According to an alternative embodiment of the system according to the present invention, layer 6 does not present the features set forth above, and it is an impermeable material layer of the type with which the tubular container 2 is made.

System 1 comprises a recirculation system 7 of the percolate within the tubular container 2 for favouring the production of biogas during the anaerobic processing step.

The recirculation system 7 comprises a plurality of percolate collection tubes 8 arranged near a bottom 2c of the tubular container 2, a plurality of percolate introduction tubes 9 arranged near the upper portion 2b of the container 2, and a pump 10. Obviously, the tubes 8 and 9 are appropriately perforated.

In this way, the percolate inside the biomass can be aspirated by the tubes 8 by the action of the pump 10 and reintroduced within the biomass itself through tubes 9 to favour the production of biogas.

System 1 comprises a recirculation system 11 of air adapted to ensure appropriate oxygenation during the aerobic step.

The recirculation system 11 comprises a plurality of air intakes 12 arranged near a bottom 2c of the container 2, a plurality of air intake tubes 13 arranged between the upper portion 2b of the container 2 and the adsorbing and transpiring layer 6 and a fan 14 for recirculating the air. Obviously, tubes 12 and 13 are appropriately perforated.

During the aerobic treatment step, the intake tubes 13 have a two-fold purpose: the first is to take exhausted air from the waste mass being stabilised and to introduce it into the heap, the second is to ensure a closed-circuit system operation without the emission of gas and vapour outside.

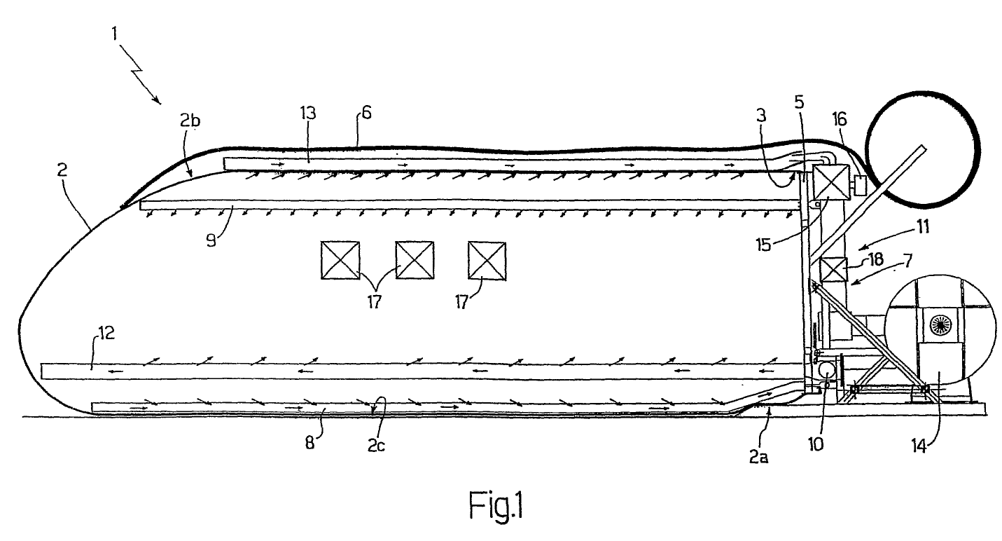

System 1 comprises a device 15 (schematically shown in FIG. 1) for extracting air from outside the tubular container 2, which is connected to the air circulation system 11. Device 15 is provided with a valve (known and not shown), for connecting the external air intake to the recirculation system 11 or not according to the quantity of oxygen detected in the recirculation system 11 itself.

In particular, an oxygen sensor will check the percentage of oxygen present in the air circulating the recirculation system 11, and when the latter drops under a predetermined limit the valve for mixing the exhaust air with the external air will be opened and the right oxygen mixture will be created.

Furthermore, the system 1 comprises a biogas collection device 16 (schematically shown in FIG. 1) connected to the air recirculation system 11 and adapted to collect the biogas formed following the anaerobic treatment step.

Finally, the system 1 comprises a plurality of thermometers 17 (schematically shown in FIG. 1) arranged within the tubular container 2, and a hygrometer 18 (schematically shown in FIG. 1) accommodated and operating in the recirculation system 11.

The system 1 comprises a control unit known and not shown which may be controlled via the Internet and which is capable of managing the various parts of the system.

In use, the waste to be processed, after being appropriately prepared by possible shredding and sieving, as well as mixed with appropriate structuring material, if required, is charged with inoculation liquid for boosting the fermentation process, and then introduced in the tubular container 2 by a typical bagging machine normally used for farming applications such as those used for bagging fodder and products intended for feeding animals.

Once the tubular container 2 is filled, the opening through which the waste was introduced is closed, the percolate recirculation system 7 is operated and the conditions for producing biogas, i.e. for performing the anaerobic process step, are created within the tubular container 2.

The anaerobic processing step lasts three to four weeks during which a biogas is produced that will be collected by device 16. The collected biogas will be subjected to a first pre-treatment step related to dehydration and purification and then will be introduced in the cogeneration plant.

After the first anaerobic processing step, openings 4 are made at the upper portion 2b. At this point, the most possible percolate which can be aspirated is aspirated from within the tubular container by tubes 8 and recovered in a tank, and the air recirculation system 11 is put into operation. In these conditions, the anaerobic processing step will last for approximately from two to three weeks.

During this aerobic process step, according to the treatment conditions, either the complete sanitation and bio-stabilisation of the waste heap or the formation of compost is obtained.

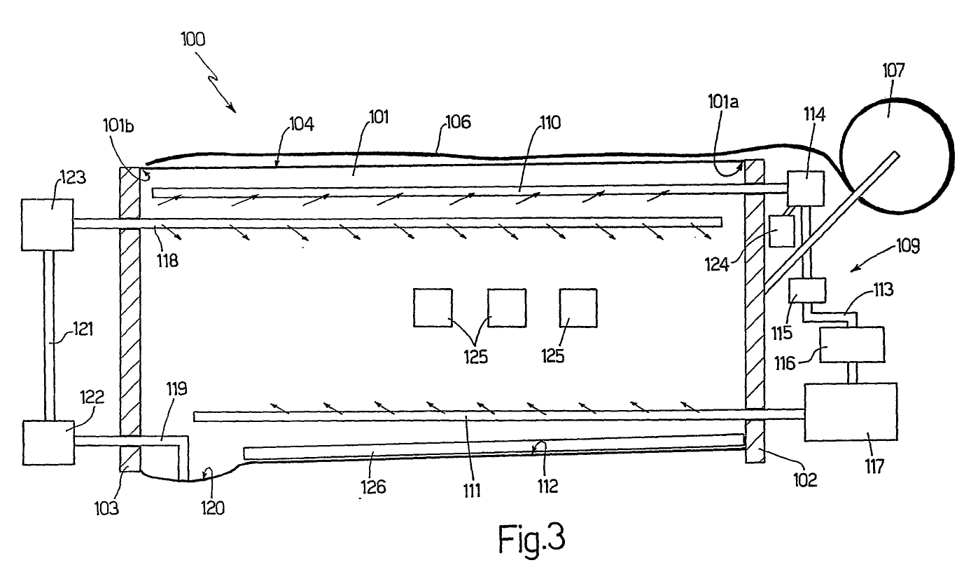

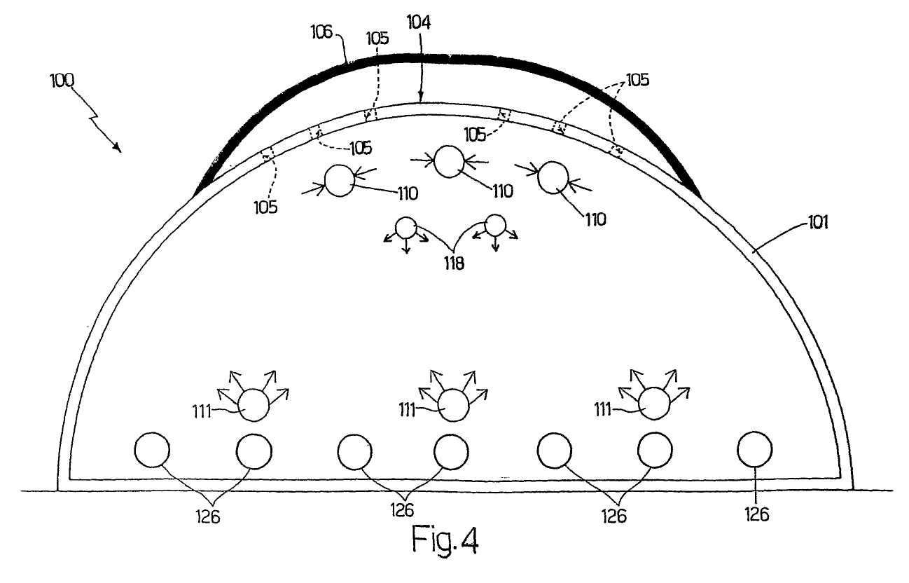

In FIGS. 3 and 4, it is indicated by 100 a further embodiment of the system according to the present invention. System 100 comprises a tubular container 101 made of polyethylene or other suitable material and presenting two ends 101a and 101b closed by respective plates 102 and 103 made of metallic material. As previously specified for system 1, the tubular container 101 is also made of non-rigid material which facilitates transportation, installation and removal thereof.

System 100 comprises an adsorbing and transpiring layer 106 arranged at an upper portion 104 of the tubular container 101 to close openings 105 formed as described below. Layer 106 has the same features already shown for layer 6 and is wound on a specific winder/roller 107 fastened to the plate 102.

System 100 comprises an air recirculation system 109 comprising a plurality of air intake tubes 110 arranged within the tubular container 101 near the upper portion 104 and fastened to the plate 102, a plurality of air intakes 111 arranged within the tubular container 101 near a bottom portion 112 and also fastened to the plate 102 and a union line 113 fastened to the plate 102 externally to the tubular container 101 and adapted to join tubes 110 with tubes 111. The union line 113 comprises in sequence following the flow of air and schematically illustrated in FIG. 3, a device 114 for collecting air from the outside provided with valve, an oxygen gauge 115, a dehumidifier 116 and a fan 117. Optionally, a cooling element may be arranged on union line 113 and downstream of the dehumidifier 116. As apparent, tubes 111 and 112 present a plurality of holes adapted to allow the passage of air.

System 100 comprises a percolate recirculation system comprising a plurality of perforated percolate introduction tubes 118 arranged within the tubular container 101 near its upper portion 104 and fastened to the plate 103, a plurality of collection tubes 119 arranged within the tubular container 101 fastened to the plate 103 and adapted to take by their ends from within a collection tank 120, and a union line 121 fastened to the plate 103 externally to the tubular container 101 and adapted to join tubes 118 and tubes 119. The collection tank 120 is arranged in the bottom portion 112 of the tubular container 101 and collects the percolate formed due to the presence of a plurality of draining tubes 126 resting on the bottom portion 112 arranged on an incline. The union line 121 comprises (schematically shown in FIG. 3) a pump 122 and a union 123 adapted to be connected to an external percolate source to be introduced within the tubular container 101.

As described above for system 1, system 100 comprises (schematically shown in FIG. 3) a biogas collection device 124 connected with the air recirculation system 109 and a plurality of thermometers 125.

System 100 comprises a control unit (known and not shown) which may be controlled via the Internet and which is capable of reading the values shown by the measuring instruments and of consequently controlling the various parts of the system.

The waste treatment procedure using system 100 is the same as that shown with reference to system 1, with the difference that the percolate introduced within the tubular container may also be originated outside the system and that the air recirculation collection tubes are arranged inside instead of outside the container.

The system of the present invention offers the advantage that the entire process is performed in a single system thus avoiding movements of materials with respective environmental problems.

Moreover, the system of the present invention offers the advantage of using particularly economical consumable materials without for this loosing in efficiency, and allows to perform the processing processes without logistic constraints, minimising at the same time the leakage of gas and bad odours.

Another advantage offered by the system is that it may be mobile and therefore moved to various areas of the waste treatment system according to logistics.

Moreover, the system of the present invention allows to perform treatments also outdoor or sheltered only by a lean-to roof, thus eliminating the enormous costs for purifying air within specific sheds.

Finally, the system may be used also separately, i.e. either as an anaerobic system for the production of biogas or an aerobic system for stabilisation and compositing.

1. A waste treatment system comprising:
a permeable material container adapted to accommodate waste, and presents at least one opening for introducing the waste, air-tight closing element of said opening;
a percolate introduction element within the permeable material container;
a collector for the formed biogas, a covering layer made of an adsorbing and transpiring material or an impermeable material and arranged at an upper portion of said container; and
an air recirculation system within said container.

2. A system according to claim 1, wherein said impermeable material container is made of non-rigid material and adapted to be gathered and spread according to various steps of use.

3. A system according to claim 1, wherein said air circulation system comprises a plurality of air intake tubes and a plurality of air intakes both arranged within said container.

4. A system according to claim 3, wherein said air circulation system further comprises a device for collecting air from outside the tubular container.

5. A system according to claim 4, wherein said air collection device is provided with a valve adapted to be operated according to the quantity of oxygen measured in said recirculation system.

6. A system according to claim 1, wherein said air recirculation system comprises a dehumidifying device arranged between said air intake tubes and said air intakes.

7. A system according to claim 1, wherein said percolate introduction element comprises a plurality of percolate introduction tubes arranged within the container;
a plurality of percolate collection tubes arranged within the container and adapted to take by their ends from within a collection tank, and a recirculation pump; and
said collection tank being arranged in a bottom portion of the container.

8. A system according to claim 7, comprising a connection device adapted to connect said percolate introduction tubes to a percolate source external to said system.

9. A system according to claim 1, wherein said impermeable material container has a tubular shape, and in that it comprises at least one first metallic plate arranged so as to close the impermeable container.

10. A system according to claim 9, wherein said air recirculation system is fastened to said metallic plate.

11. A system according to claim 10, comprising a second metallic plate also arranged so as to close the impermeable container on the opposite side with respect to said first metallic plate and on which said percolate introduction element are fastened.

12. A system according to claim 1, wherein said air recirculation system comprises a plurality of air intake tubes arranged between said impermeable container and said covering layer, and a plurality of air intakes arranged within said container.

13. A system according to claim 12, wherein said air circulation system comprises a device for collecting air from outside the tubular container.

14. A system according to claim 13, wherein said air collection device is provided with a valve adapted to be operated according to the quantity of oxygen measured in said recirculation system.

15. A system according to claim 1, comprising a percolate recirculation system comprising a plurality of percolate collection tubes arranged near a bottom portion of the tubular container, a plurality of percolate introduction tubes arranged near the upper portion of the container, and a recirculation pump.
